(19) United States
(12) Patent Application Publication
(10) Pub. No.: US 2010/0209394 A1
(43) Pub. Date: Aug. 19, 2010

(54) MITOGEN ACTIVATED PROTEIN KINASE MODULATOR

(75) Inventors: Rajiv Indravadan Modi, Ahmedabad (IN); Devesh Bhardwaj, Ahmedabad (IN); Nirav M. Desai, Ahmedabad (IN); Bakulesh Mafatlal Khamar, Ahmedabad (IN)

Correspondence Address: DARBY & DARBY P.C., P.O. BOX 770, Church Street Station, New York, NY, 10008-0770, US

(73) Assignee: CADILA PHARMACEUTICALS LTD., Ahmedabad (IN)

(21) Appl. No.: 12/666,852

(22) PCT Filed: Jun. 26, 2008
(86) PCT No.: PCT/IB08/01675

§ 371 Date: Jan. 14, 2010

(30) Foreign Application Priority Data
Jun. 28, 2007 (IN) 1235/MUM/2007

(51) Int. Cl. A61K 35/74; C12N 1/20; A61P 29/00; A61P 43/00

(52) U.S. Cl. 424/93.4; 435/253.1

(57) ABSTRACT

The present invention describes the method of modulating MAPK pathways. Further more the invention describes the use of Mycobacterium w for modulation of MAPK pathway intermediates for treatment of MAPK mediated conditions.

### FIELD OF THE INVENTION

This invention relates to tailored modulation of MAP kinases using Mycobacterium w (Mw) and/or its constituents.

Further it relates to means for transient or sustained modulation of MAP kinases.

### BACKGROUND OF THE INVENTION

The mitogen-activated protein kinase (MAPK) cascade is a major signaling system that is shared by various types of cells. They are serine/threonine kinases. They translocate on activation (phosphorylation) into nucleus. They phosphorylate/activate many different proteins including transcription factors including that regulate expression of important cell-cycle and differentiation specific proteins. The genes regulated are involved in apoptosis, inflammation, cell growth, and differentiation.

These proteins mediate varieties of cellular responses and biological activities including morphogenesis, cell death, stress responses, immune responses, cell proliferation, apoptosis, paraapoptosis, cell survival etc.

Activation of MAPK cascade is not restricted to immature cells, and this cascade is also activated in terminally differentiated cells such as neutrophils, suggesting that the MAPK cascade also plays an important role in some functions of terminally differentiated mature cells.

Activation of MAP kinase in two different cells can lead to similar or different cellular responses. The ERK cascade is activated in response to signals from receptor tyrosine kinases, hematopoietic growth factor receptors, or some heterotrimeric G-protein-coupled receptors and appears to mediate signals promoting cell proliferation or differentiation.

The stress activated protein kinase (SAPK), includes p38 and JNk, is activated in response to heat shock, hyperosmolarity, UV irradiation, protein synthesis inhibitors or inflammatory cytokines and appear to be involved in the cell responses to stresses.

Activation of the distinct MAPK subtype cascade is dependent on the types of cells and the stimuli used. The functional role of each MAPK subtype may be different according to the types of cells.

Belmont et al. describe means for treating a JNK mediated disorders by administering to a subject in need thereof an effective dose of a therapeutic agent that modulates (inhibits or enhances, as required) the activity of JNK. Agents that stimulate a JNK signal transduction pathway can be used in a number of ways, including inducing programmed cell death (apoptosis) in tissues. For example, the elimination of UV damaged cells can be used to prevent cancer.

Moreover, MAPK modulators are useful in management of melanomas (Clin Cancer Res. 2006 April 1: 2371s-2375s). MAPK modulators can have synergistic action with Paclitaxol (Mol Pharmacol. 2001 August; 60(2):290-301. MAPK modulator are associated with programmed cell death (J. Biol Chem. 2000 Dec. 15;275(50):38953-6).

MAPK modulators works synergistically with biological therapy like antibacterial as well as chemotherapy. (Oncogene 2003:22,2034-2044). MAPK modulators are useful in re-sensitivity of resistant cells to chemotherapeutic agents. (Brit. J. Cancer 2001, 85: 1175-1184). MAPK modulator is associated with chemotherapeutic effects of cancer chemotherapy like Taxol, Cisplatin (Oncogene 2001;20,147-155; Onco gene 2000, 19; 5142-5152)

Activation of ERK has been primarily associated with cell growth and survival, by and large, activation of SAPK have been linked to the induction of apoptosis. Using many cell types, it was shown that persistent activation of JNK induces cell death, and that the blockade of JNK activation by dominant-negative (DN) inhibitors prevents killing by an array of apoptotic stimuli. Elevated levels of extracellular regulatory kinase (MAPK/ERK) actively are frequently found in some cancer cells. Up-regulation of IL-2 production by p38 MAPK inactivation is mediated by increased ERK1/2 activity.

ERK2 is a widely distributed protein kinase that achieves maximum activity when both Thrl83 and Tyrl85 are phosphorylated by the upstream MAP kinase kinase, MEK1 (Anderson et al., 1990, Nature 343,651; Crews et al., 1992, Science 258,478). Upon activation, ERK2 phosphorylates many regulatory proteins, including the protein kinases Rsk90 (Bjorbaek et al., 1995, J. Biol. Chem. 270,18848) and MAPKAP2 (Rouse et al., 1994, Cell 78,1027), and transcription factors such as ATF2 (Raingeaud et al., 1996, Mol. Cell Biol. 16,1247), Elk-1 (Raingeaud et al. 1996), c-Fos (Chen et al., 1993 Proc. Natl. Acad. Sci. USA 90,10952), and c-Myc (Oliver et al., 1995, Proc. Soc. Exp. Biol. Med. 210,162). ERK2 is also a downstream target of the Ras/Raf dependent pathways (Moodie et al., 1993, Science 260,1658) and may help relay the signals from these potentially oncogenic proteins. ERK2 has been shown to play a role in the negative growth control of breast cancer cells (Frey and Mulder, 1997, Cancer Res. 57,628) and hyperexpression of ERK2 in human breast cancer has been reported (Sivaraman et al., 1997, J Clin. Invest. 99,1478). Activated ERK2 has also been implicated in the proliferation of endothelin-stimulated airway smooth muscle cells, suggesting a role for this kinase in asthma (Whelchel et al., 1997, Am. J. Respir. Cell Mol. Biol. 16,589).

JNKs along with other MAPKs, have been implicated in having a role in mediate cellular response to cancer, thrombin-induced platelet aggregation, immunodeficiency disorders, autoimmune diseases, cell death, allergies, osteoporosis and heart disease.

A role for JNK in cardiovascular disease such as myocardial infarction or congestive heart failure has also been reported as it has been shown JNK mediates hypertrophic responses to various forms of cardiac stress [Circ. Res. 83:167-78 (1998); Circulation 97:1731-7 (1998); J. Biol. Chem. 272:28050-6 (1997); Circ. Res. 79:162-73 (1996); Circ. Res. 78:947-53 (1996); J. Clin. Invest. 97:508-14 (1996)].

It has been demonstrated that the JNK cascade also plays a role in T-cell activation, including activation of the IL-2 promoter. Thus, inhibitors of JNK may have therapeutic value in altering pathologic immune responses [J. Immunol. 162:3176-87 (1999); Eur. J. Immunol. 28:3867-77 (1998); J. Exp. Med. 186:941-53 (1997); Eur. J. Immunol. 26:989-94 (1996)].

A role for JNK activation in various cancers has also been established, suggesting the potential use of JNK inhibitors in cancer. For example, constitutively activated JNK is associated with HTLV-1 mediated tumorigenesis [Oncogene 13:135-42 (1996)]. In addition, regulation of the c-jun gene in p210 BCR-ABL transformed cells corresponds with activity of JNK, suggesting a role for JNK inhibitors in the treatment for chronic myelogenous leukemia (CML) [Blood 92:2450-60 (1998)].

JNK signaling, especially that of JNK3, has been implicated in the areas of apoptosis-driven neurodegenerative diseases such as Alzheimer's Disease, Parkinson's Disease, ALS (Amyotrophic Lateral Sclerosis), epilepsy and seizures, Huntington's Disease, traumatic brain injuries, as well as ischemic and hemorrhaging stroke.

JNK is potently activated by several chemotherapy drugs and oncogene products such as Bcr-Abl, Her-2/Neu, Src, and oncogenic Ras, but usually they have no action on ERK. Thus it is long standing need to design/produce agent which can perform a multifunctional role of activating SAPK and down regulating ERK at the same time.

The altered MAPK signaling pathways is associated with various disease conditions like neurodegenerative disorders, autoimmune diseases, tumor development and progression, resistance to chemotherapy damage following ischaemic insults are some of the conditions.

These conditions can be effectively treated with MAPK modulation. Radiation induced cancer cell death as well as chemotherapy induced cancer death is associated with modulation of MAK signaling pathways. E.g. up regulation of SAPK.

The transient up regulation of SAPK is also useful in inducing immune responses without inducing autoimmunity. Transient up regulation of SAPK is also useful in inducing death of cancer cells without affecting normal cells significantly. The transient up regulation of SAPKs are known to play role in Antigen presenting cell activation and maturation T cells and dendritic cells. Arrighi et al. described the role of phospho-p38 kinases in APC maturation (The Journal of Immunology, 2001, 166: 3837-3845). The up regulation of SAPK has an important role to play in T cell response as described by Mercedes Rincón et al, (Free Radical Biology and Medicine, Volume 28, Issue 9, 1 May 2000, Pages 1328-1337).

Down regulation of SAPK is useful in preventing damage in neurodegenerative disease. It is also useful in management of autoimmune disease, minimizing damage following ischaemic insult.

Thus a combined effect of SAPK down regulation and ERK up regulation or at least no change would be useful in preventing damage to the tissues in various conditions described earlier.

U.S. Pat. No. 6,994,981 describe modulators of para-apoptosis and related methods. EP1208748, WO 2004089929 & WO2006117567 are prior art patents based on MAPK inhibitors.

U.S. Pat. No. 6,852,740 B2 describe pyrazole derivatives as p38 kinase inhibitors. WO 95/31451 describes pyrazole compositions that inhibit MAPKs, and, in particular, p38. However, the efficacy of these inhibitors in vivo is still being investigated.

There is a negative cross-talk relationship between the stress-activated pathway and the mitogen-activated ERK pathway. Some of the biological functions of JNK activators, such as TNF-α and ceramide, may be attributed to their ability to block cell responses to growth and survival factors acting through the ERK/MAPK pathway hence controlling one of these results in further distortion of the signal transduction and worsens the conditions.

For most of the MAPK mediated diseased conditions require an agent which acts on MAPK modulation by regulating cross talk between SAPK and ERK.

Therefore for the treatment of such conditions wherein one or more intermediates of MAPK pathway levels or/and their ratios are erratic. Especially in case of conditions wherein more then one intermediate levels are wrongly modulated, one would need MAPK modulator.

Toxic side effect of these synthetic MAPK inhibitors are diarrhea, rash, fatigue, hand-foot syndrome, alopecia, nausea, hand-foot skin reaction, or acral erythema, characterized by painful symmetrical erythematous and edematous areas on the palms and soles, commonly accompanied by paraesthesias. Sometimes the lateral sides of the fingers or the periungual zones can be affected. Hyperkeratosis and desquamation commonly occur. Stomatitis, alopecia, pruritus, and subungual splinter hemorrhages are also observed, these hemorrhages are characterized by straight black or red lines under the nails. It seems that they originate from thrombotic or embolic mechanisms. Initially thought to be a typical sign of bacterial endocarditis, they were subsequently reported to be also present in different settings, such as antiphospholipid syndrome, severe rheumatoid arthritis, thromboangeitis obliterans, mitral stenosis, at high altitude, or when arterial catheters are used (The Oncologist, Vol. 12, No. 12, 1443-1455, December 2007;)

BCG and CpG are able to up regulate the SAPK with concurrent ERK up regulation is also observed. The ERK up regulation leads to the TNF and IL-10, up regulation, which are inflammatory cytokines leading to side effects.

Therefore there is a long felt need to develop MAPK modulators that are useful in treating various conditions associated with erratic levels SAPKs and ERK, which is non-toxic.

In the present invention a novel approach is taken to modulate signal transduction pathways through the MAPK/ERK intermediate levels modulation, wherein MAPK/ERK pathway is modulated/interrupted by Mycobacterium w depending on the dose of Mw and status of cell, hence having low toxic. Also Mycobacterium w formulation is already used for different indication for treatment of more then a million human patients, and has proved safe.

### SUMMARY OF THE INVENTION

The object of the invention is to provide the modulator for the Mitogen activated protein kinase (MAPK) signal transduction pathway.

Another object of the invention is to provide the modulation of mitogen activated protein kinase using Mycobacterium w and/or its constituents.

Another object of the invention is to provide a method for modulating Mitogen activated protein kinases.

Yet another object of the invention is to provide Mitogen activated protein kinase modulator, where in SAPK are down regulated while levels of ERK shows no change.

Yet another object of the invention is to provide Mitogen activated protein kinase modulator, where in SAPK are up regulated while levels of ERK are down regulated.

Yet another object of the invention is to provide Mitogen activated protein kinase modulator, where in SAPK are up regulated while levels of ERK shows no change.

Yet another object of the invention is to provide MAPKs modulator administered through parenteral, entral and topical route in mammals.

### BRIEF DESCRIPTION OF THE DRAWINGS

FIG. 1. Down regulation of SAPK with no change in ERK: In vitro

FIG. 2. Down regulation of SAPK with no change in ERK: In vivo

FIG. 3. Tailored effect of MAPK modulation using different Mw concentrations.

FIG. 4. Up regulation of SAPK with down regulation of ERK: in vitro

FIG. 5. Tailored effect of MAPK modulation using different Mw concentrations.

FIG. 6. Longevity of MAPK modulation by Mw

FIG. 7. Transient up regulation of SAPK with no change in ERK levels

### DETAIL DESCRIPTION OF THE DRAWINGS

In accordance with the present invention the MAPK modulators are prepared by the following process. The invention includes the composition of pharmaceutical composition, the method of preparation, HPLC characteristic, its safety and tolerability, methods of use and outcome of treatments are described in following examples. The following are illustrative examples of the present invention and scope of the present invention should not be limited by them.

### The Process of Preparing a Mycobacterium w:

### A. Culturing of Mycobacterium w.

i) Preparation of culture medium.

Mycobacterium w is cultured on solid medium like L J medium or liquid medium like middle brook medium or Sauton's liquid medium.

For better yield middle brook medium is enriched. It can be preferably enriched by addition of glucose, bacto-tryptone and BSA. They are used in ratio of 20:30:2 preferably.

The enrichment medium is added to middle brook medium. It is done preferably in ratio of 15:1 to 25:1 more preferably in ratio of 20:1.

ii) Bioreactor operation

a) Preparation of vessel:

The inner contact parts of the vessel (Joints, mechanical seals, o-ring/gasket grooves, etc.) should be properly cleaned to avoid any contamination. Fill up the vessel with 0.1 N NaOH and leave as such for 24 H to remove pyrogenic materials and other contaminants. The vessel is then cleaned first with acidified water, then with ordinary water. Finally, the vessel is rinsed with distilled water (3 times) before preparing medium.

b) Sterilization of bioreactor:

The bioreactor containing 9 L distilled water is sterilized with live steam (indirect). Similarly the bioreactor is sterilized once more with Middle brook medium. The other addition bottles, inlet/outlet air filters etc. are autoclaved (twice) at 121° C. for 15 minutes. Before use, these are dried at 50° C. oven.

c) Environmental parameters
i. Temperature: 37±0.5° C.
ii. pH: 6.7 to 6.8 initially.
   B. Harvesting and concentrating:

It is typically done at the end of 6^{th} day after culturing under aseptic condition. The concentration of cells (palletisation) is done by centrifugation.

C. Washing of cells:

The pallet so obtained is washed minimum three times with normal saline. It can be washed with any other fluid which is preferably isotonic.

D. Adding pharmaceutically acceptable carrier.

(Pyrogen free normal saline is added to pallet) Any pyrogen free isotonic fluid can be used as a pharmaceutical carrier, most preferably. The carrier is added in amount so as get to desired concentration of active in final form.

E. Additing preservative:

Adding preservative to keep the cell/pellets free from contamination. Preferably thiomesol is used having concentration of 0.01% w/v.

### F. Terminal Sterilization:

Sterilizing the cell/pallet by various physical methods like application of heat or ionizing radiation or sterile filtration. Heat can be in the form of dry heat or moist heat. It can also be in the form of boiling or pasturisation. Ionizing radiation can be ultraviolet or gamma rays or mircrowave or any other form.

G. Quality control:
i. The material is evaluated for purity, sterility.
ii. The organisms are checked for acid fastness after gram staining.
iii. Biochemical Test: The organism is subjected to following biochemical tests:
   a) Urease
   b) Tween 80 hydrolysis
   c) Niacin test
   d) Nitrate reduction test
      The organism gives negative results in urease, tween 80 hydrolysis and niacin test. It is positive by nitrate reduction test.
iv. Inactivation test: this is done by culturing the product on L J medium to find out any living organism.
v. Pathogenicity and/or contamination with pathogen
   The cultured organisms are infected to balb/c mice. None of the mice should die and all should remain healthy and gain weight. There should not be any macroscopic or microscopic lesions seen in liver, lung, spleen, or any other organs when animals are sacrificed upto eight weeks following treatment.
      H. Preparation of Mycobacterium w constituents:

Mw constituents can be prepared by following methods.
I. Cell disruption
II. Solvent extraction
III. Enzymatic extraction.

The cell disruption can be done by sonication or using of high pressure fractionometer or application of osmotic pressure ingredient. The disrupted cells were washed with physiological saline and re-paleted by centrifugation.

The solvent extraction can be done by any organic solvent like chloroform, ethanol, methanol, acetone, phenol, halogenated hydrocarbons isopropyl alcohol, acetic acid, urea, hexane and/or aromatic compound individually or in any combination thereof can do the solvent extraction.

The enzymatic extraction can be done by proteolytic enzymes which can digest cell wall/membranes. Lysozymes, Liticase and pronase are the preferred enzymes. Mw cell constituents can be used in place of Mw. Addition of Mw cell constituents results in improved efficacy of the product.

The cultured organisms are infected to Balb/c mice. None of the mice should die and all should remain healthy and gain weight. There should not be any macroscopic or microscopic lesions seen in liver, lung spleen or any other organs when animals are killed upto 8 weeks following treatment.

The Mycobacterium w so prepared was evaluated for its MAPK modulating activity. Following examples are illustrative of MAPK modulation by Mycobacterium w and/or its constituents.

Following examples demonstrate the invention and are not limiting the scope of the invention.

A. NFS 60 cell line: Sample Preparation

The cell pellet of 1*10̂7 cells, stored after harvesting are processed as follows: The cells are solubilized in lysis buffer # 6 (1 mM EDTA, 0.5% Triton X-100, 6 M Urea, 10 μg/mL Leupeptin, 10 μg/mL Pepstatin, 100 μM PMSF, 3 μg/mL Aprotinin, 2 mM sodium pyrophosphate, 1 mM activated sodium orthovanadate in PBS, pH 7.2-7.4)
The lysate is briefly vortexed and allowed to sit on ice for 10 min. It is then centrifuged at 10,000 rpm for 10 min in a plastocraft. The supernate is transferred to fresh tubes. A 6 fold dilution of the lysate is prepared with IC Diluent # 8 (1 mM EDTA, 0.5% Triton X-100 in PBS, pH 7.2-7.4) and further dilution is done in IC Diluent # 3. The sample in IC Diluent #3 is used for analysis by ELISA using kits from R & D Systems

B. ELISA Plate Preparation:
Phospho-P38 (R & D Systems, DuoSet IC Cat # DYC869-2)
Phospho-JNK (R & D Systems, DuoSet IC Cat # DYC1018-2)
Phospho-JNK (R & D Systems, DuoSet IC Cat # DYC1387-2)

The Capture Antibody was diluted to the working concentration according to the manufacturer's instruction. 96 well microplates immediately coated with 100 μL per well of the respective diluted Capture Antibody. The plates were sealed and incubated overnight at room temperature.

The plates were aspirated and washed with Wash Buffer repeating the process two times for a total of 3 washes. After the last wash any remaining Wash Buffer was removed by inverting the plate and blotting it against clean blotting papers. The plates were blocked by adding 300 μL of Block Buffer to each well. The plates were then incubated at room temperature for 2 hrs after which the plates were again washed thrice, aspirated and tapped dry. The plates are now ready for sample addition. The strips for the respective MAPK were stored in a dessicator kept at 2-8° C. for further use.

C. Splenocyte Preparation:

Splenocytes were harvested from normal mice. The mice were sacrificed by cervical dislocation. The abdominal cavities were immediately opened and the spleens were isolated. Each spleen was individually processed further.

Each spleen was washed clean of contaminating blood and other impurities with 10 ml of Dulbeccos's Phosphate Buffered Saline (DPBS). The spleens were chopped using a sterile syringe piston. 10 ml of RPMI-1640 complete media (10% FBS and 1% Penicillin-Streptomycin antibiotic) was added and the contents transferred to a 50 ml falcon tube by sieving the chopped spleens through a 40 μm nylon cell strainer. The cells were pelleted by centrifugation at 1500 rpm for 5 minutes in a Hereaeus Multifuge-3 SR.

The supernatant was discarded and the RBC's present in the pellet were lysed by re-suspending the cells in 5 ml of Lysis buffer (0.144 M NH₄Cl in 0.017M Tris-HCl at pH32 7.6) for 10 minutes. The reaction was stopped by the addition of 40 ml DPBS. The cells were pelleted by centrifugation at 1500 rpm for 5 minutes in a Hereaeus Multifuge-3 SR. The pellet of each set of cells obtained was re-suspended in 1 ml of RPMI-1640 complete media. Thereafter the cell number was determined using a Neubarr chamber by staining the cells with trypan blue. The cell suspension was diluted appropriately with RPMI-1640 complete media to obtain a cell density of 10̂7 cells/ml.

From each spleen a 5 mL suspension containing 1*10̂7 cells/ml in RPMI-1640 was prepared. 1 ml of each of the respective cell suspension was seeded in micro titre plates in quadruplets, of which 2 were stimulated with 10̂8 cells of Mycobacterium w. The plate was incubated for 36 hrs at 37° C. at 6% CO₂. After 48 hours cells were harvested. The cells after thorough pipetting were transferred to eppendorf tubes. The cells were pelletd by centrifugation at 10,000 rpm for 10 min. The supernate was transferred to separate tubes. Both the supernates and cell pellets were labeled and stored at −70° C. until further analysis.

### Example 1

Down regulation of SAPK with no Change in ERK:

In vitro effect of Mw:

Splenocytes were isolated from naïve Balb/C mice and cultured in RPMI 1640 media with 10% FBS and 1% antibiotics in microtiter plate. The cells were divided in two sets each of 10̂6/mL splenocytes: The set one was incubated with PBS (control) set 2 was incubated with 10̂8 Mycobacterium w cells.

At 48 hrs of culture the cells were harvested and the MAPK ELISA (phospho-JNK, phospho-p38, and phospho-ERK assays) were performed as per manufacturer's instructions, using the commercial kit as described above from R & D Systems.

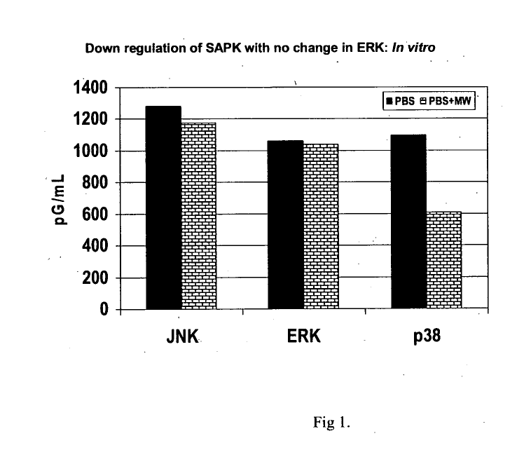

The Phospho-JNK results were plotted as indicated in FIG. 1. It is observed that in vitro stimulation of Splenocytes with 10̂8 Mycobacterium w does not show any significant change in Phospho-JNK levels over 48 hrs.

The phospho-p38 results were plotted as indicated in FIG. 1. It is observed that in vitro stimulation of Splenocytes with Mycobacterium w down regulates phospho-p38 MAPK after 48 hrs with 10̂8 Mycobacterium w cells Thus Mycobacterium w down regulates phospho-p38 levels.

The Phospho-ERK results were plotted as indicated in FIG. 1. It is observed that in vitro stimulation of Splenocytes with Mycobacterium w does not show any significant change in phospho-ERK levels over 48 hrs compared to the control.

In vivo effect of Mw:

Splenocytes were isolated from Balb/C mice on day-7 after they were immunized with 0.1 mL of PBS in group one, and group two received 0.1 mL Mycobacterium w (10̂8 cells) intradermally. The cells were cultured in RPMI 1640 media with 10% FBS and 1% antibiotics in microtiter plate.

At 48 hrs of culture the cells were harvested and MAPK ELISA (phospho-JNK, phospho-p38, and phospho-ERK assays) were performed as per manufacturer's instructions, using the commercial kit as described above from R & D Systems.

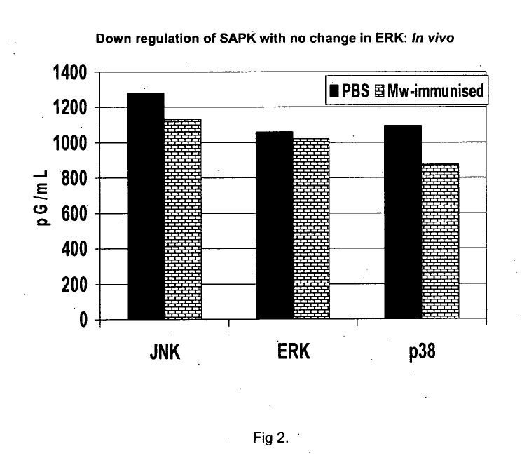

The results are depicted in FIG. 2. It was observed that in mice immunized with Mw shows down regulation of SAPKs while no change in ERK levels.

Tailored effect using different Mw concentrations:

Splenocytes were isolated from naïve Balb/C mice and cultured in RPMI 1640 media with 10% FBS and 1% antibiotics in microtiter plate. The cells were divided in three sets each of 10̂6/mL splenocytes. The set one was incubated with PBS (control) set 2 was incubated with 10̂8 Mycobacterium w cells and set 3 was incubated with 10̂6 Mycobacterium w cells.

At 48 hrs of culture the cells were harvested and the MAPK ELISA (phospho-JNK, phospho-p38, and phospho-ERK assays) were performed as per manufacturer's instructions, using the commercial kit as described above from R & D Systems.

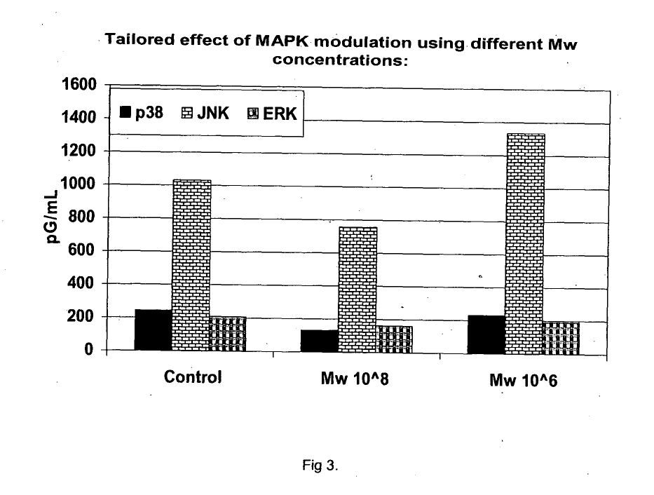

The Phospho-JNK results were plotted as indicated in FIG. 3. It is observed that in vitro stimulation of Splenocytes with 10̂6 Mycobacterium w does not show down regulation in Phospho-JNK levels over 48 hrs, while when stimulated with 10̂8 Mycobacterium w cells shows down regulation of Phospho-JNK levels at the end of 48 hrs. In 1998 Dong, Chen et al described in, Science 282: 2092-2095 and Yang, D. D, described in Immunity 9: 575-585, that Phospho-JNK up regulation is required for T cell differentiation, proliferation and activation.

The phospho-p38 results were plotted as indicated in FIG. 3. It is observed that in vitro stimulation of Splenocytes with Mycobacterium w down regulates phospho-p38 MAPK after 48 hrs with 10̂8 Mycobacterium w cells and not 10̂6 Mycobacterium w cells. Arrighi et al. described the role of phospho-p38 kinases in APC maturation (The Journal of Immunology, 2001, 166: 3837-3845).

The Phospho-ERK results were plotted as indicated in FIG. 3. It is observed that in vitro stimulation of Splenocytes with Mycobacterium w does not show any significant change in phospho-ERK levels over 48 hrs compared to the control.

### Example 2

Up Regulation of SAPK with Down Regulation in ERK:

In vitro effect of Mw:

NFS 60 cells were cultured in DMEM media with 10% FBS, 1% antibiotics and IL-3 10 nG/mL. The cells were plated in micro titer wells at concentration of 1×10̂5 cells. The numbers of wells were divided in to two sets. Set one was incubated with PBS as control, and two with 3×10̂7, Mycobacterium w cells.

At 1, 2, 4, 8, and 24 hrs of culture the cells were harvested and MAPK ELISA (phospho-JNK, phospho-p38, and phospho-ERK assays) were performed as per manufacturer's instructions, using the commercial kit as described above from R & D Systems.

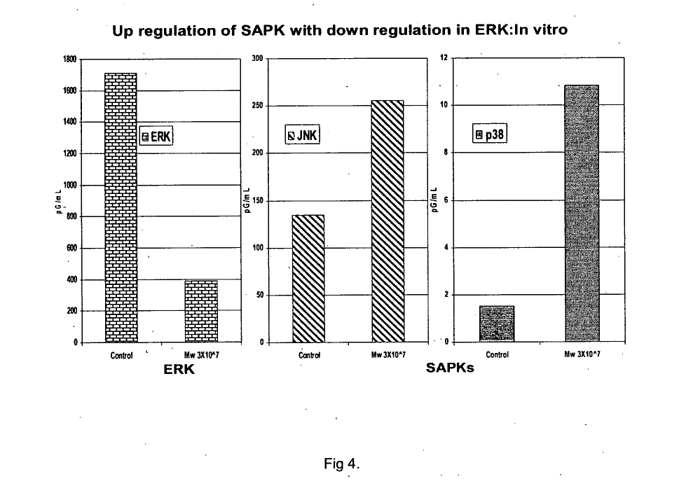

The phospho-JNK levels in Mycobacterium w stimulated cells compared to control shows no significant change till 4 hrs and at the end of 8^{th} to 24^{th} hrs it becomes double FIG. 4.

The phospho-p38 levels in Mycobacterium w stimulated cells compared to control shows differential dose dependent effect. At dose of 3×10̂7 Mycobacterium w cells the phospho-p38 levels shoots up and remains high for 4 to 8 hrs then starts dropping down but at the end of 24 hrs they are lower then the control (FIG. 4). It was surprising that at concentration of 3×10̂7 Mycobacterium w cells for stimulation the NFS-60 cell are killed.

The phospho-ERK levels in control shows no significant change till 8 hrs and at the end of 24 hrs it becomes double. This may be due to the increase in cell numbers. While the Mycobacterium w stimulated cells shows initial at 1 hr it self increase in phospho-ERK levels to double and at 8^{th} hrs it drops to half of 1 hrs and at 24 hrs the levels are almost ¼ of control at that time as shown in FIG. 4. The drop was associated with the decrease in live cell numbers.

Thus rise in SAPKs (phospho-p38 and phospho-JNK levels) with concurrent drop of phospho-ERK levels are associated with the death of NFS60 cells.

Tailored effect using different Mw concentrations:

NFS 60 cells were cultured in DMEM media with 10% FBS, 1% antibiotics and IL-3 10 nG/mL. The cells were plated in micro titer wells at concentration of 1×10̂5 cells. The numbers of wells were divided in to five sets. Set one was incubated with PBS as control, and remaining each with 3×10̂7, 1×10̂7, 7×10̂6, and 3×10̂6 Mycobacterium w cells respectively.

At 1, 2, 4, 8, and 24 hrs of culture the cells were harvested and MAPK ELISA (phospho-JNK, phospho-p38, and phospho-ERK assays) were performed as per manufacturer's instructions, using the commercial kit as described above from R & D Systems.

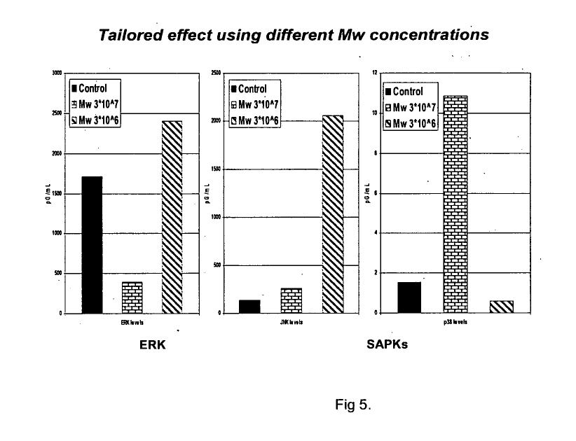

The phospho-JNK levels in 3×10̂7 Mycobacterium w stimulated cells compared to control shows no significant change till 4 hrs and at the end of 8^{th} to 24^{th} hrs it becomes double FIG. 5 while at lower concentration the rise in levels are delayed to no effect in study period (FIG. 5).

The phospho-p38 levels in Mycobacterium w stimulated cells compared to control shows differential dose dependent effect. At dose of 3×10̂7 Mycobacterium w cells the phospho-p38 levels shoots up and remains high for 4 to 8 hrs then starts dropping down but at the end of 24 hrs they are lower then the control. With all the other Mycobacterium w concentrations the phospho-p38 levels are down regulated as in control (FIG. 5).

The phospho-ERK levels in control shows no significant change till 8 hrs and at the end of 24 hrs it becomes double. This may be due to the increase in cell numbers. While the Mycobacterium w stimulated cells at 8^{th} hrs shows drop to half of 1 hrs and at 24 hrs the levels are almost ¼ of control at that time as shown in FIG. 5. The drop was associated with the decrease in live cell numbers.

### Example 3

### Longevity of MAPK Modulation:

Splenocytes were isolated from Balb/C mice immunized with 1 mL of PBS in group one, group two to six received 1 mL Mycobacterium w (10̂9 cells) intravenous. The group 1 and 2 were sacrificed on day 1, while group three on 7 day, group four on 14 day, group five on 21 day, group six on 28 day and cultured in RPMI 1640 media with 10% FBS and 1% antibiotics in microtiter plate. The cells were divided in three sets each of 10̂6/mL splenocytes. The set one was incubated with PBS (control) set 2 was incubated with 10̂8 Mycobacterium w cells and set 3 was incubated with 10̂ Mycobacterium w cells.

After 48 hrs cells were harvested and the MAPK ELISA (phospho-JNK, phospho-p38, and phospho-ERK assays) were performed as per manufacturer's instructions, using the commercial kits as described above from R & D Systems.

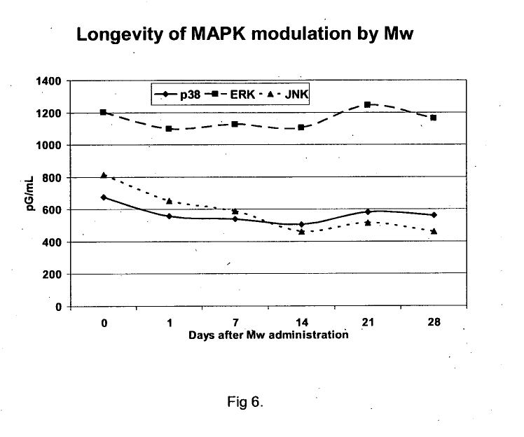

The phospho-JNK levels show down regulation from 24 hrs after immunization till 28^{th} days (period of study) as shown in FIG. 6.

The phospho-p38 levels also showed down regulation from 24 hrs after immunization but till 21^{th} days as shown in FIG. 6.

The phospho-ERK levels show down regulation from 24 hrs after immunization till 14^{th} days after which it regains normal levels as shown in FIG. 6.

### Example 4

Transient Up regulation of SAPKs with no Change in ERK:

Splenocytes were isolated from naïve Balb/C mice and cultured in RPMI 1640 media with 10% FBS and 1% antibiotics, in microtiter plate. The cells were divided in three sets each of 10̂6/mL splenocytes. The set one was incubated with PBS (control) set 2 was incubated with 10̂8 Mycobacterium w cells and set 3 was incubated with 10̂6 Mycobacterium w cells.

At 1, 2, 4, 8, and 24 hrs of culture the cells were harvested and the MAPK ELISA (phospho-JNK, phospho-p38, and phospho-ERK assays) were performed as per manufacturer's instructions, using the commercial kit as described above from R & D Systems.

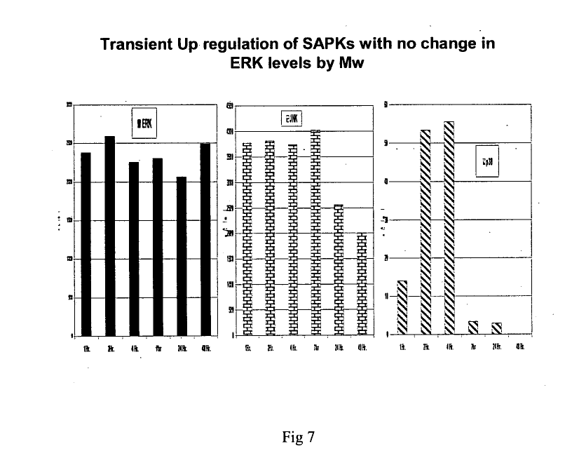

The phospho-JNK levels measured by ELISA shows up-regulation of phosphor phospho-JNK with 10̂6 Mycobacterium w. The up regulation is transient only between 1 to 8 hrs while at 24^{th} hrs the levels are down regulated as shown in FIG. 7.

The phospho-p38 levels measured by ELISA shows up-regulation of phosphor phospho-p38 with 10̂8. The up regulation is transient only between 1 to 4 hrs while from 8^{th} to 24^{th} hrs the levels are down regulated as shown in FIG. 7.

The up regulation of SAPK has an important role to play in T cell response as described by Mercedes Rincón et al,(Free Radical Biology and Medicine, Volume 28, Issue 9, 1 May 2000, Pages 1328-1337). These findings are dose dependent as the lower levels, 10̂6 cell, of the Mycobacterium w do not exhibit this properties as shown in FIG. 7, while in ERK such modulation was not observed.

We claim:

1. Mycobacterium w (Mw) and/or constituents thereof are modulators of Mitogen activated protein kinases in mammal and/or mammalian cells.

2. A method of modulating Mitogen activated protein kinases in a mammal in need thereof, said method comprising the step of administering Mycobacterium w and/or constituents thereof.

3. A method of modulating Mitogen activated protein kinases as claimed in claim 2 wherein the concentration of Mycobacterium w and/or constituents thereof used is in the range of 10̂12 to 10̂1 of Mycobacterium w cells.

4. A method of modulating Mitogen activated protein kinases as claimed in claim 2 wherein the preferred concentration of Mycobacterium w and/or constituents thereof used is in the range of 10̂8 to 10̂6 of Mycobacterium w cells.

5. Modulation of Mitogen activated protein kinases as claimed in claim 2 comprises transient up regulation of SAPK with no change of ERK levels.

6. Modulation of Mitogen activated protein kinases as claimed in claim 2 comprises transient up regulation of SAPK with down regulation of ERK levels.

7. Modulation of Mitogen activated protein kinases as claimed in claim 2 comprises down regulation of SAPK with no significant alteration of ERK levels.

8. Modulation of Mitogen activated protein kinases as claimed in claim 2 wherein modulation is determined by changing an amount of Mycobacterium w and/or its constituents.
